(19) United States
(12) Patent Application Publication
(10) Pub. No.: US 2024/0399326 A1
(43) Pub. Date: Dec. 5, 2024

(54) FIXED-BED TUBULAR REACTOR COMPRISING A SEPARATIVE MEMBRANE

(71) Applicant: COMMISSARIAT A L'ENERGIE ATOMIQUE ET AUX ENERGIES ALTERNATIVES, Paris (FR)

(72) Inventors: Frédéric DUCROS, Grenoble Cedex 09 (FR); Isabelle CHAMPON, Grenoble Cedex 09 (FR)

(73) Assignee: COMMISSARIAT A L'ENERGIE ATOMIQUE ET AUX ENERGIES ALTERNATIVES, Paris (FR)

(21) Appl. No.: 18/698,219

(22) PCT Filed: Oct. 3, 2022
(86) PCT No.: PCT/FR2022/051863

§ 371 Date: Apr. 3, 2024

(30) Foreign Application Priority Data
Oct. 5, 2021 (FR) FR2110519

(51) Int. Cl. B01J 8/02; B01J 8/06; C07C 29/152

(52) U.S. Cl. CPC B01J 8/0214; B01J 8/065; C07C 29/152; B01J 2208/00884; B01J 2208/00938

(57) ABSTRACT

A fixed-bed tubular reactor that extends between first and second ends and includes a bed of catalyst powder confined in an annular space located between an outer wall of a hollow tube and an inner wall of a hollow insert, which comprises a distribution chamber and a collection chamber. The inner wall is covered with a permselective membrane allowing partial removal of at least one reaction product, in that it comprises at least one make-up chamber separated from the at least one distribution chamber and collecting chamber by at least one first dividing wall. The at least one make-up chamber includes an inlet port for at least one make-up fluid and an outlet port for the at least one make-up fluid.

### TECHNICAL FIELD

The present invention is directed to the general field of exchange reactors, and more particularly with the field of catalytic exchange reactors using a solid catalyst, especially in powder form, and for implementing endothermic or mainly exothermic catalytic reactions.

Such reactions can especially be implemented for the synthesis of fuels and combustibles, for example liquid fuels, such as methanol (MeOH), or gas fuels, such as methane or Synthetic Natural Gas (SNG), dimethyl ether (DME), hydrocarbons or olefins, obtained from hydrogen and carbon oxides or from synthesis gas comprising a mixture of hydrogen and carbon oxides. They may also relate to the generation of hydrogen from methane, the synthesis of ammonia from hydrogen and nitrogen, or hydrogenation and dehydrogenation reactions involving hydrogen and molecules of the Liquid Organic Hydrogen Carrier (LOHC) type.

The invention can thus be more particularly applied to highly exothermic reactions, such as methanation (or more generally hydrocarbon production or hydrogenation reactions) of carbon monoxide or carbon dioxide in the presence of hydrogen. It can also be applied to Fischer-Tropsch type reactions, wet or dry reforming of methane or other hydrocarbons, or oxidation or dehydrogenation reactions. The invention can also be used as a heat exchanger for applications, especially those implementing a gas, which require frequent maintenance operations, for example due to corrosion, fouling, among other things.

The invention thus provides a fixed-bed tubular reactor capable of implementing especially exothermic or endothermic organic synthesis methods.

### State of Prior Art

Catalytic reactors using solid catalysts are widely used for the synthesis of organic compounds such as synthetic fuels or combustibles, including Synthetic Natural Gas (SNG), dimethyl ether, methanol, hydrocarbons or even olefins.

Within the scope of the production of hydrocarbons, or more generally for hydrogenation reactions, from hydrogen and carbon oxide, the equilibria involved, or hereafter referred to as the main reactions, are generically as follows:

$$n CO + ( 2 n + 1 ) {H}_{2} ⇔ {C}_{n} {H}_{2 n + 2} + {n H}_{2} O \qquad Eq . 1$$

$${n CO}_{2} + ( 3 n + 1 ) {H}_{2} ⇔ {C}_{n} {H}_{2 n + 2} + 2 {n H}_{2} O \qquad Eq . 2$$

$$X + {n H}_{2} ⇔ ( - {X H}_{2 n} - ) \qquad Eq . 3$$

The equilibrium described by the third equation (Eq. 3) relates to molecules of the liquid organic hydrogen carrier (LOHC) type. This is a reversible reaction, which is thus successively implemented in exothermic and then endothermic sequences. These various reactions are well documented in thermochemical terms. They potentially give rise to side reactions (e.g. WGS (Water Gas Shift), RWGS (Reverse Water Gas Shift), Boudouard formation, etc.) known to those skilled in the art and not described here.

Species involved in these main reactions and side reactions are called “reactants” for species entering the reactor, and “products” for species produced by the main and side reactions. In addition, “reactive fluids” refers to all the species involved in these reactions. These reactions are all associated with thermodynamic conditions, pressure and temperature, which are preferential given the performance targets: it is generally interesting to work at high pressure and it is also necessary to correctly manage exothermicity of these reactions by extracting heat from the reaction zone, which makes it possible to guarantee the conversion rate, selectivity and catalyst life time, among other things. Other functionalities discussed below are also of interest.

A number of catalytic reactor architectures used in industry for the thermal control and regulation of endothermic or exothermic chemical reactions are already known. Main types of heat exchanger reactors known for exothermic reactions are described below.

Firstly, the simplest catalytic reactor technology is the so-called “fixed-bed” reactor technology. In cascaded adiabatic fixed-bed reactors, exothermicity is generally managed by diluting reactants at the inlet to the first reaction stage, for example by recirculating the products, and by installing heat exchangers for cooling the reactant-product mixture between the different reactors. This architecture has the advantage of being simple to manufacture, but requires the installation of gas recirculation systems to limit the rise in temperature and imposes the use of catalysts that are stable at high temperatures. These reactors tend to be used as centralised units operating under steady-state conditions.

There is also the technology of fluidised-bed heat exchanger reactors. These reactors were developed to solve the problem of heat transfer in fixed-beds. These reactors offer the advantage of a good thermal homogeneity in the reactor, which avoids hot spots, but require a larger reactor volume for equivalent power than in the case of fixed plug-flow beds. In these reactors, the catalyst is in the form of fine particles whose attrition has to be controlled. In addition, fluidisation of particles means that the gas flow rate variation ranges have to be limited, which makes these reactors inflexible for intermittent operation.

Another exchanger reactor technology relates to exchanger reactors in which the chemical reaction takes place within a reactive channel that is continuously cooled by an external heat transfer fluid. Most of these reactors are of the calender-tube type, with the reaction taking place in reaction tubes cooled at the periphery by a heat transfer bath. The reactive gases axially circulate in the tubes, which contain a catalyst, for example in powder form.

The combination of heat exchanger reactors of the same type or of different types within a same unit can also be contemplated in order to improve conversion, flexibility or reuse of the heat recovered.

The management of thermal stresses on reactors begins with the response to the need for thermal control. This can be achieved in a number of ways, as set out in the previous technological solutions, i.e. solutions outside the reactor, such as staggering the overall conversion, with intermediate cooling and/or dilution and/or condensation; solutions inside the reactor, such as moving towards the concept of heat exchanger reactors, boosting heat exchange, reducing the size of reactive channels (millistructuring), integrating 3D conductive structures for thermal homogenisation, and staggering reactant injections for distributing the energy deposition.

As for the question of catalyst life time, it should be noted that exothermic reactions generate a large amount of heat, which can lead to the appearance of hot spots resulting in local degradation of the catalyst and degradation of the conversion performance of the reactor/catalyst assembly. In other words, degradation of the solid catalyst can lead to its deactivation and a reduction in the conversion rate of the chemical species present. The selectivity of the reactions involved is also altered. Furthermore, some hydrogenation reactions that are very useful in industry, such as methanol synthesis or Fischer-Tropsch synthesis, as described by the previous equations Eq.1 to Eq.3, are equilibrated and have low to moderate conversion rates for the thermodynamic conditions usually retained. An interesting strategy thereby consists in associating permselective membranes with one or more of the walls of the reactive channels, so as to subtract one of the species produced, typically water, from the reactive medium, thereby disequilibrating the reaction and increasing its productivity. The advantages of such a method have been demonstrated both experimentally and theoretically. Innovations can focus on two main elements: membranes and reactor integration methods.

In the conventional solution of a heat exchanger reactor with the catalyst situated in the tubes, for tube-calendar type technologies, one of the problems is controlling the zones that become hotter due to the exothermicity of the reaction. This phenomenon requires the tubes to be intensively cooled over their entire surface area, whereas at any given time only a small part of this surface area needs to be intensively cooled, for example for injection and axial circulation of the reactants. One consequence of this is that the thermal fluid flow rate is oversized.

To overcome these problems, an arrangement which distributes reactants over the entire length of the tubes has been provided. This solution makes it possible to obtain a more uniform temperature over the entire length of the reactor. In this respect, documents U.S. Pat. Nos. 3,758,279 A, 4,374,094 A, EP 0 560 157 A1 and 2,997,374 A provide heat exchanger reactors using a distribution of reactants from an annular distributing space. Especially, these exchange reactors, which are generally cylindrical in shape, comprise, being coaxially arranged and from outside of the reactor, a tube, the annular distribution space, a catalyst load and a collection space. These solutions are the standard for cooling in tube and calender type reactors, i.e. the cooling of the tubes is regulated solely by flow in the calender.

However, these arrangements are not satisfactory. Indeed, the presence of the annular distribution space disposed around the catalyst load limits heat transfer from the catalyst to the tube, making inefficient the cooling systems generally considered. It remains possible, however, to insert heat-conducting elements into the reactor. However, such a solution is incompatible with reactors comprising small-diameter tubes.

Conversely, document CN 103990420 A suggests implementing an insert with a distribution chamber and a collection chamber, disposed in the centre of a tube and defining with the latter an annular space housing the solid catalyst. However, the arrangement provided in that document does not allow homogeneous distribution within the annular space. More particularly, this arrangement does not allow an optimum temperature profile to be obtained within the solid catalyst.

FIG. 1 in document U.S. Pat. No. 8,961,909 A represents another example of a calender-tube type reactor. This reactor is especially equipped with an injection tube, immersed in a catalytic powder bed, and along which apertures are provided. The latter are especially arranged to ensure injection of the reactive gas at different levels of the catalytic powder bed, and thus limit the appearance of hot spots in said bed. However, this reactor is not satisfactory. Indeed, in order to ensure its cooling, this reactor requires the installation of a plurality of heat transfer fluid circulation circuits, which further increases its complexity.

Document U.S. Pat. No. 7,402,719 B2, especially FIG. 3a, discloses another example of a reactor arranged to allow staggered injection of a reactant C with a view to its reaction with a reactant A. This reactor comprises two webs (or channels) separated by a wall and intended to ensure circulation of reactant A and reactant C respectively. The two webs are additionally in fluid communication by means of a plurality of apertures provided in the wall separating them. These apertures are especially arranged to ensure gradual mixing of reactant C with reactant A. This gradual mixing thus limits the appearance of hot spots. However, the fact that the reactor is arranged in the form of a stack of webs means that it is not very compact.

Furthermore, in the Applicant's patent application FR 3 103 714 A1, an insert-type device organises axial distribution of the reactive gases in the tubes, circulation of the gases in a substantially orthoradial direction through a bed of catalyst in an annular space defined by the inner wall of a tube and the outer wall of the insert, and axial collection of the gases produced.

There is still a need to improve these types of reactors, and especially to provide such tube/calendar reactors with one alternative fluid circulation allowing integration of a separative element and management of the fluid for discharging the chemical species separated.

### DISCLOSURE OF THE INVENTION

The purpose of the invention is therefore to remedy, at least in part, the needs mentioned previously and the drawbacks relating to embodiments of prior art.

The invention is especially intended to provide a fixed-bed tubular reactor allowing more uniform distribution of the reactants within the solid catalyst, more uniform distribution of the heat flow generated within the solid catalyst, and better cooling management. In addition, the invention seeks to provide a tubular reactor for which the reliability and life time (of the catalysts) are improved compared with reactors known from the state of the art, and which makes it possible to optimise (increase) the gas passage time into the fixed-bed of catalyst powder.

One object of the invention is thus, according to one of its aspects, a fixed-bed tubular reactor which extends, along a longitudinal axis, between a first end and a second end, said reactor comprising a catalytic powder bed confined in an annular space situated between an outer wall of a hollow tube and an inner wall of a hollow insert disposed coaxially in the hollow tube, the hollow insert comprising at least one distribution chamber and at least one collection chamber, separated from each other by at least one first separative wall, said at least one distribution chamber and said at least one collection chamber comprising a gas intake opening at the first end, and a gas discharge opening at the second end, respectively,
characterised in that the inner wall of the hollow insert is covered, especially partially or completely, with a separative structure comprising at least one permselective membrane for partially removing at least one reaction product, so that the annular space is delimited by the outer wall and the permselective membrane,
and in that the reactor additionally includes at least one supply chamber separated from said at least one distribution chamber and from said at least one collection chamber by at least one first separative wall, said at least one supply chamber comprising an inlet port for at least one supply fluid consisting of a flushing fluid for discharging said at least one reaction product, distinct from the gases circulating in said at least one distribution chamber and in said at least one collection chamber, at the first end, and an outlet port for said at least one supply fluid, at the second end.

By virtue of the invention, it is especially possible to improve cooling of the reaction zone (reactants, reaction products and catalysts) by enabling this zone to be cooled by an internal and external circulation of heat transfer fluid, which is beneficial for all hydrogenation reactions, for example. In addition, it can improve the conversion rate of equilibrated reactions such as the synthesis of methanol, olefins, hydrocarbons or even the hydrogenation of molecules of the liquid organic hydrogen carrier (LOHC) type.

The reactor according to the invention may further include one or more of the following characteristics taken in isolation or according to any possible technical combinations.

The permselective membrane may be organic or inorganic. It may advantageously be conformable. It may be as described in the book “In-Situ H₂O removal via hydrophilic membranes during Fischer-Tropsch and other fuel-related synthesis reactions”, M. P. Rhode, Phd Dissertation, KIT Scientific Publishing, 2010.

The permeability of the permselective membrane can advantageously be selectively exerted with respect to water vapour.

According to one alternative, the separative structure can additionally include a porous support covering the inner wall of the insert, the porous support itself being covered with the permselective membrane, and the hollow insert can include apertures to allow said at least one reaction product to be separated from said gases to pass therethrough.

According to another alternative, the hollow insert can be made of a porous material, to allow said at least one reaction product to be separated from said gases to pass therethrough, and covered with the permselective membrane, and said at least one first separative wall of the hollow insert can include a sealing, especially ceramic, material, for sealing with respect to the reactive gases.

Each of said at least one distribution chamber, at least one collection chamber and at least one supply chamber can be delimited by a section of the inner wall and two first separative walls.

In addition, the reactor may include at the first end and at the second end, respectively, a distributing space and a collecting space between which the hollow insert is disposed.

Furthermore, the tube and the insert can be held by at least two tubular holding plates respectively at the first and second ends, at least one of said at least two tubular holding plates, especially all the tubular holding plates, including an inlet or outlet duct respectively fluidically connected to a supply fluid inlet or outlet port and formed in said at least one of said at least two tubular holding plates, especially to allow side intake and/or extraction of supply fluid.

The axial lengths of the tube and insert may be different, the axial length of the tube being especially shorter than the axial length of the insert.

Alternatively, the tube and the insert can be held by at least two tubular holding plates at the first and second ends respectively, and at least one inlet or outlet duct fluidically connected to an inlet or outlet port for supply fluid can be situated in the distributing space and in the collecting space respectively, outside said at least two tubular holding plates, especially to allow side intake and/or extraction of supply fluid.

Further alternatively, the tube and the insert can be held by at least two tubular holding plates at the first and second ends respectively, and said inlet port and said outlet port for said at least one supply fluid can be formed in the upper and lower ends of the insert respectively to allow feed from the distributing space and extraction from the collecting space respectively, the reactor further comprising at least one side gas feed duct and at least one side gas extraction duct.

Said at least one side feed duct and the feed of supply fluid from the distributing space on the one hand, and said at least one side extraction duct and the extraction of supply fluid from the collecting space on the other hand, can be separated by a sealed separation plate.

Furthermore, a seal can be disposed between the insert and each of the sealed separation plates.

Furthermore, the flow of said at least one supply fluid can be made co-currently or counter-currently to the flow of gases in said at least one distribution and collection chamber.

If necessary, the catalytic powder can be retained in the annular space by a seal of fibrous material at each of the ends of the annular space.

In addition, the insert can be a one-piece part.

Additionally, among other objects, another object of the invention is the use of a tubular reactor as defined previously, characterised in that it implements endothermic or exothermic reactions for synthesising fuels and combustibles.

### BRIEF DESCRIPTION OF THE FIGURES

The invention will be better understood upon reading the following detailed description, of non-limiting exemplary implementations thereof, as well as upon examining the schematic and partial figures of the appended drawing, in which:

FIG. 1 is a partial schematic representation of a fixed-bed tubular reactor in accordance with the invention, along a cross-sectional plane passing through the longitudinal axis of the reactor, in particular along the longitudinal cross-sectional plane PP of FIG. 2, for viewing a collection chamber and a distribution chamber,

FIG. 2 is a cross-section view along a transverse plane, or normal cross-section, perpendicular to the longitudinal axis of the tubular reactor of FIG. 1,

FIG. 2A is a view similar to FIG. 2, for viewing a first alternative of using a separative structure according to the principle of the invention,

FIG. 2B is a view similar to FIG. 2, for viewing a second alternative of using a separative structure according to the principle of the invention,

FIG. 3 is a schematic representation of the tubular reactor of FIGS. 1 and 2 along the longitudinal cross-section plane P′P′ of FIG. 2, for viewing two supply chambers fed via side inlet and outlet ports,

FIG. 4 is a view similar to that of FIG. 1, for illustrating the use of seals to retain the catalyst,

FIG. 5 is a view similar to that of FIG. 3, for illustrating the use of seals to retain the catalyst,

FIG. 6 is a schematic representation of another example of a tubular reactor in accordance with the invention, in a view similar to that of FIG. 1,

FIG. 7 is a view similar to that of FIG. 3 for the reactor of FIG. 6,

FIG. 8 is a schematic representation of another example of a tubular reactor in accordance with the invention, according to a view similar to that of FIG. 1,

FIG. 9 is a view similar to that of FIG. 3 for the reactor of FIG. 8,

FIGS. 10 and 11 are axial cross-section views, respectively along planes PP and P′P′ with reference to FIG. 2, of a plurality of tubular reactors similar to that of FIGS. 1 to 5, situated inside a calender,

FIG. 12 is an axial cross-section view, along the plane P′P′ with reference to FIG. 2, of a plurality of tubular reactors similar to those in FIGS. 6 and 7, situated inside a calender,

FIG. 13 is a graph representing the course of the conversion of carbon dioxide as a function of the arc length of the insert, in a configuration with the presence of a membrane and in a configuration without the presence of a membrane, and

FIG. 14 is a graph representing a cross-section of the catalyst showing the course of the molar concentrations and the total water (H₂O) flow lines.

Throughout these figures, identical references may designate identical or analogous elements.

In addition, the different parts represented in the figures are not necessarily drawn to a uniform scale, to make the figures more legible.

### DETAILED DESCRIPTION OF PARTICULAR EMBODIMENTS

The present invention relates to a tubular heat exchanger reactor with a fixed catalytic powder bed. In particular, the catalytic powder bed is confined in an annular space situated between one wall, referred to as the outer wall, of a hollow tube and another wall, referred to as the inner wall, of a hollow insert coaxially housed in said tube. The catalytic powder bed can especially include a catalyst as grains.

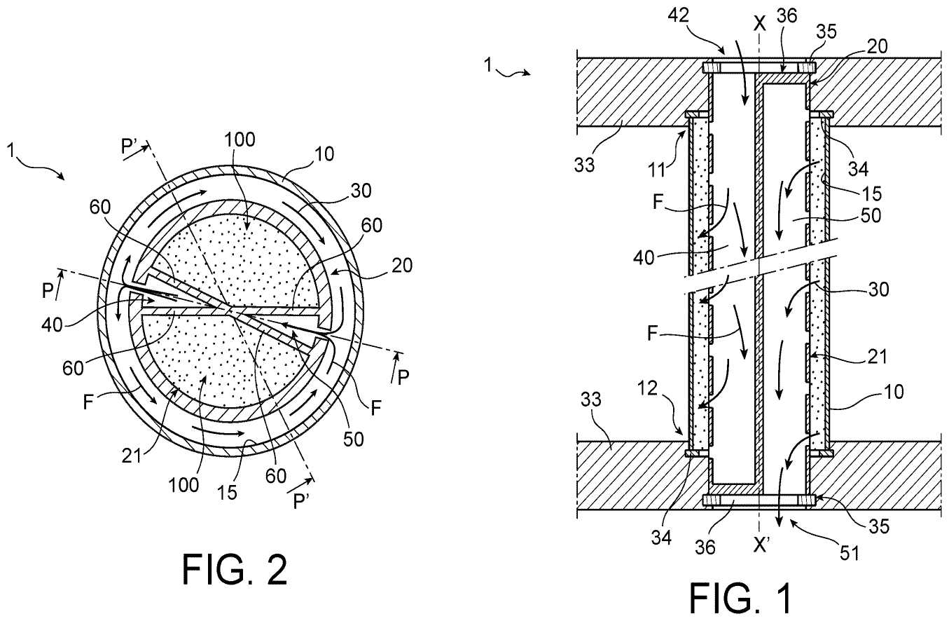

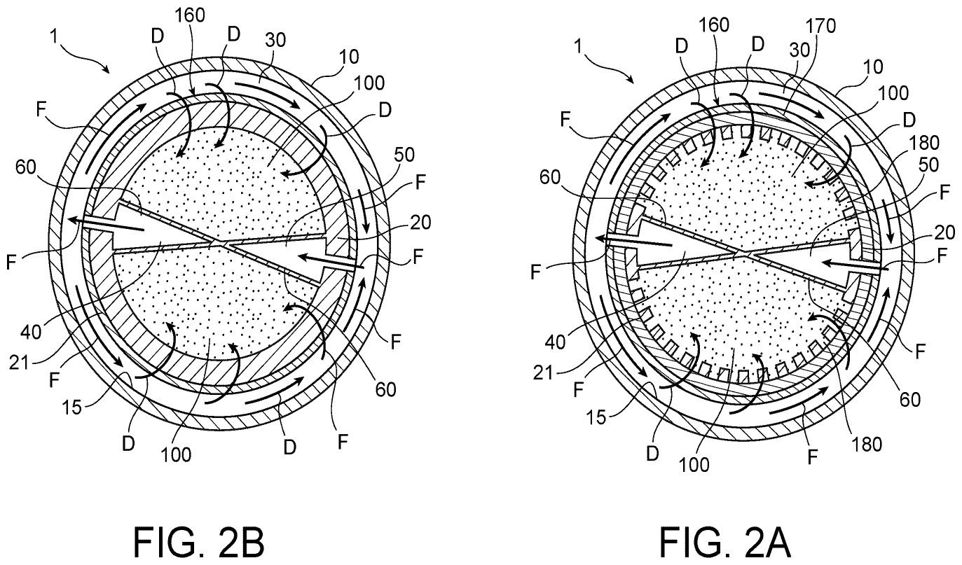

It is to be noted that in all FIGS. 1, 2 and 3 to 12, the separative structure comprising a permselective membrane 160 according to the principle of the invention is not represented. It is therefore appropriate to refer to FIGS. 2A and 2B, which illustrate this principle. It should also be noted that the alternative embodiments of the separative structure described with reference to FIGS. 2A and 2B are applicable to the embodiments of FIGS. 1, 2 and 3 to 12.

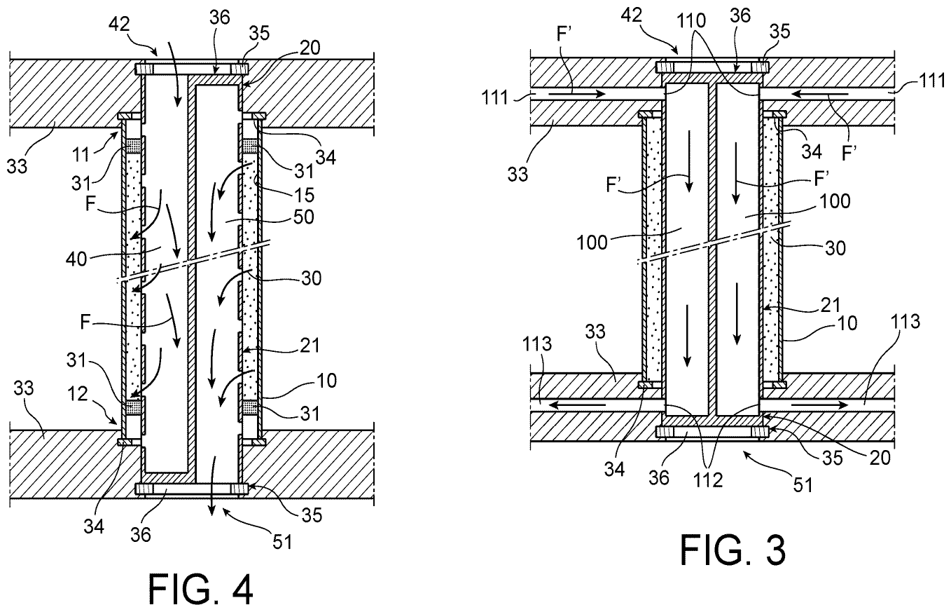

Thus, in FIGS. 1 and 2 one exemplary embodiment of a fixed-bed tubular reactor according to the present invention can be seen. It is to be noted that in these FIGS. 1 and 2, as well as in all the figures described below, the arrows F represent the direction of travel of the gas. On the other hand, the arrows F′, especially visible in FIGS. 3, 5, 7, 9 and 11, represent the direction of travel of the supply fluid.

The tubular reactor 1 according to the present invention comprises an outer hollow tube 10 which extends along a longitudinal axis XX′ between a first end 11 and a second end 12. The hollow tube 10 may have a symmetry of revolution about the longitudinal axis XX′. It is therefore understood that the longitudinal axis XX′ may be an axis of revolution of the hollow tube 10.

The hollow tube 10 may comprise a metal, especially a metal selected from: steel, aluminium alloy, copper, nickel, among others. The diameter of the hollow tube 10 may comprise between 5 mm and 100 mm. The wall, called the outer wall 15, forming the hollow tube 10 may have a thickness of between 0.5 mm and 10 mm. The hollow tube 10 may have a length between 10 and 200 times the diameter of the inner surface.

The tubular reactor 1 also comprises a hollow insert 20 which also extends along the longitudinal axis XX′ and has a generally cylindrical shape. The hollow insert 20 is especially housed in the volume of the hollow tube 10 coaxially with the same. In particular, the insert 20 also comprises a wall, called the inner wall 21, in particular a gas-permeable wall, which delimits an annular space 30 with the outer wall 15. The annular space 30 is, in this respect, filled with a catalytic powder, and will be the place for the reactions for converting reactive gases likely to pass through the tubular reactor 1. The annular space 30 may have a thickness, defined as the distance between the outer wall 15 and the inner wall 21, of between 2% and 20% of the diameter of the inner surface of the hollow tube 10. The hollow insert 20 may be a one-piece part. The hollow insert 20 may, for example, be made of stainless steel, especially by soldering methods, or of aluminium, especially by extrusion or additive manufacturing methods (e.g. a 3D manufacturing method), or even of polymers for some low-temperature reactions. The various openings in the hollow insert 20 can be made during manufacture of the insert, and/or made by machining at a later stage.

The hollow insert 20 also comprises at least one distribution chamber 40 and at least one collection chamber 50. Here, for the sake of simplicity, a single distribution chamber 40 with a single injection point and a single collection chamber 50 are represented, but this choice is not restrictive. In particular, the hollow insert 20 may comprise between 1 and 4 distribution chambers 40, and between 1 and 4 collection chambers 50.

Said at least one distribution chamber 40 and said at least one collection chamber 50 are advantageously disposed alternately, and extend over the entire length of the hollow insert 20.

Advantageously, the hollow insert 20 also comprises at least one supply chamber 100 organising the axial circulation of a supply fluid, herein two supply chambers 100, but the invention is not limited as to the number of additional supply chambers and fluids.

Thus, by observing a plane in cross-section normal to the axis XX′ of the hollow tube 10, as visible in FIG. 2, there are successively a first supply chamber 100, said at least one collection chamber 50, a second supply chamber 100 and said at least one distribution chamber 40. Said at least one collection chamber 50, said at least one distribution chamber 40 and the supply chambers 100 are furthermore separated from each other by first separative walls 60. It is therefore understood that a distribution chamber 40 is delimited by two separative walls 60 and a section of the inner wall 21. Equivalently, a collection chamber 50 is also delimited by two first separative walls 60 and another section of the inner wall 21. Still equivalently, a supply chamber 100 is separated by two first separative walls 60 and yet another section of the inner wall 21.

Furthermore, the first dividing walls 60 extend along the entire length of the hollow insert 20 in the volume defined by the hollow tube 10, and are arranged to prevent any direct passage of gas from one chamber to the other. For example, the first separative walls 60 form planes passing through the longitudinal axis XX′.

In particular, the two first dividing walls 60 of a distribution chamber 40 may have a generally elongate shape and extend along the longitudinal axis XX′ from the first end 11 towards the second end 12. In particular, the two first separative walls 60 of a distribution chamber 40 may have a common side coinciding with the longitudinal axis XX′.

Furthermore, the reactor 1 may include, at the first end 11 of the hollow insert 20, a distributing space 42, or inlet plenum, through which one or more reactive gases are likely to be taken into the distribution chamber 40 via an intake opening. Similarly, the reactor 1 may include, at the second end 12 of the hollow insert 20, a collecting space 51, or outlet plenum, through which one or more gases are likely to be discharged through a discharge opening.

In addition, the distribution chamber 40 is shuttered at the second end 12, and the collection chamber 50 is shuttered at the first end 11.

The inner wall 21 can further comprise at least one distributing opening and at least one collecting opening, allowing respectively the distribution of a gas likely to be taken in via the intake opening at the inlet plenum 42 into a distribution compartment towards the annular space 30, and the collection of the gas distributed in the annular space 30 via the collection chamber 50.

The hollow tube 10 is advantageously held by two tubular holding plates 33, each of which includes a first system for sealingly attaching 34 the hollow tube 10 to each holding plate 33. Similarly, the hollow insert 20 is advantageously held by the two holding plates 33, each of which includes a second system 35 for sealingly attaching the hollow insert 20 to each holding plate 33. Separation walls 36 are also present in each of the holding plates 33, between which the hollow tube 10 and the hollow insert 20 are contained.

In accordance with the invention, and as visible in FIGS. 2A and 2B, the inner wall 21 of the hollow insert 20 is covered, wholly or partially, with a separative structure 160, 170 comprising a permselective membrane 160 for partially removing a reaction product and improving productivity of the chemical reaction, so that the annular space 30 is delimited by the outer wall 15 and the permselective membrane 160.

Advantageously, but not restrictively, the permeability of the permselective membrane 160 is selectively exerted with respect to water vapour H₂O.

The addition of such a permselective membrane 160 advantageously makes it possible to create a disequilibrium in the chemical reaction which is beneficial to the performance of the assembly. The membrane 160 may be organic or inorganic, and preferably shapeable.

This membrane 160 can be used to take off part of the water vapour H₂O, considering that the supply fluid F′ is a flushing fluid which discharges species to be removed, in this case the water vapour H₂O. The collection chamber 50 includes an a priori residual H₂O water vapour concentration.

FIGS. 2A and 2B represent two alternative embodiments of the selective structure for extracting the reaction product, in this case water vapour H₂O. In FIGS. 2A and 2B, the arrows D represent diffusion through the membrane 160.

In the example of FIG. 2A, the separative structure additionally includes a porous support 170 covering the inner wall 21 of the insert 20. This porous support 170 is itself covered with the permselective membrane 160. In addition, the hollow insert 20 includes apertures 180 to allow water vapour H₂O to pass therethrough.

In the example of FIG. 2B, the hollow insert 20 is made of a porous material to allow water vapour H₂O to pass therethrough, and is covered with the permselective membrane 160. In addition, each first separative wall 60 of the hollow insert 20 includes a sealing, especially ceramic, material for sealing against reactive gases.

Thus, the invention advantageously takes advantage of the use of supply chambers 100 to allow circulation and collection of the species to be separated, here water vapour H₂O.

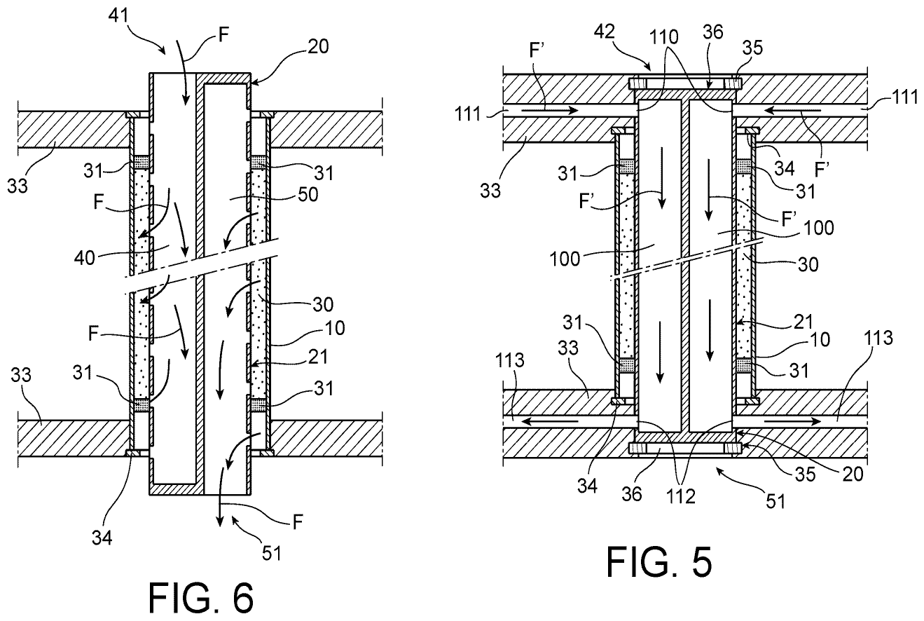

According to one advantageous aspect illustrated in FIGS. 4 and 5, the catalytic powder is retained in the annular space 30 by a seal 31, for example made of fibrous material, at each of the ends of the annular space 30. Insofar as the seal 31 is made of fibrous material, the latter is necessarily porous and therefore permeable to the reactive gases. In this respect, the fibrous material may comprise at least one of the elements chosen from: glass fibre, ceramic fibre, metal fibre, carbon fibre or polymer material fibre.

The seal 31 may especially be in the form of a braid, a sheath, a cord or simply comprise a stuffing of the fibrous material. Advantageously, the fibrous material is a thermal insulator and has a thermal conductivity substantially equivalent to that of the catalyst used (0.2 W/m/K to 10 W/m/K).

FIG. 3 is for viewing an axial cross-section along the plane P′P′ of FIG. 2, in the supply chambers 100 described previously. Each supply chamber 100 is fed by a side inlet port 110, situated in proximity to the end 11 of the hollow tube 10, this inlet port 110 being fed by a side inlet duct 111 formed in the upper holding plate 33.

Similarly, each supply chamber 100 includes a side outlet port 112, situated in proximity to the end 12 of the hollow tube 10, this outlet port 112 being fluidly connected to a side outlet duct 113 formed in the lower holding plate 33. In this way, a supply fluid F′ can be taken by circulation into the tubular holding plates 33.

Advantageously, the invention thus makes it possible to extend thermalisation capacities of reactor 1, in particular to make it possible to manage and circulate supply fluids, especially utility or reactive fluids, in addition to the reaction reactants and products, for example according to equations Eq. 1 and/or Eq. 2 described previously.

The geometry of the hollow insert 20 and the geometry of the hollow outer tube 10 are defined so as to allow separate feed of the distribution and supply chambers 40 and 100, and separate outlets from the collection 50 and supply 100 chambers.

In the exemplary embodiment of FIGS. 1 to 5, the hollow tube 10 and the hollow insert 20 have different axial lengths, the hollow tube 10 being shorter than the hollow insert 20, and the supply fluid is fed and discharged through a holding plate 33, via ducts 111 and 113. In addition, the flow of the supply fluid F′ is directed axially and co-currently to the gas circulation. However, a counter-current concept may also be of interest.

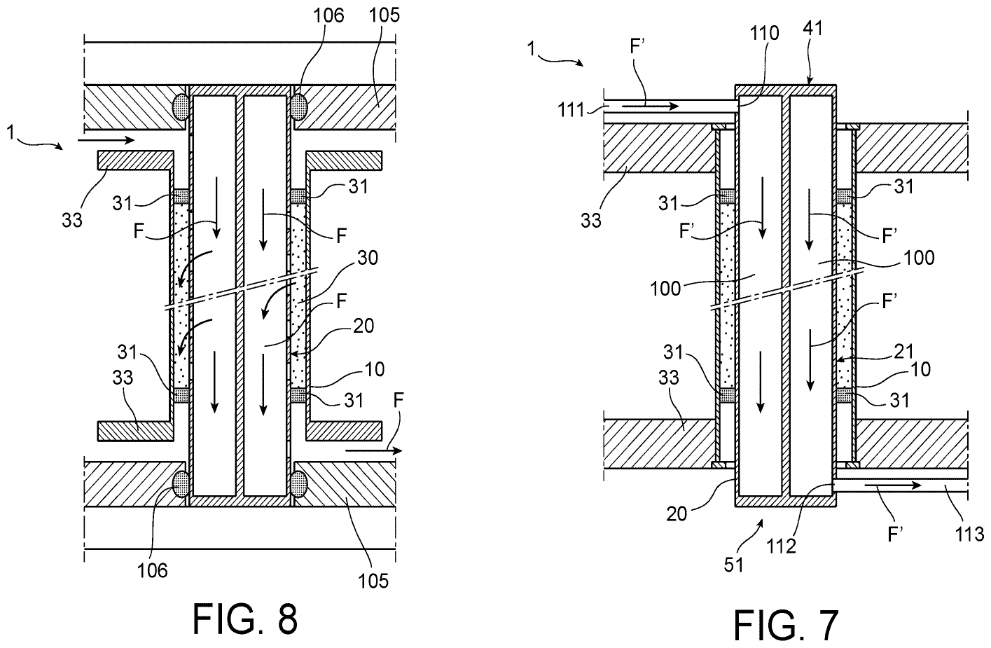

In the exemplary embodiment of FIGS. 6 and 7, the supply chambers 100 are fed via dedicated ducts situated in the inlet 42 and outlet 51 plenums, and no longer via machined ducts in the holding plates 33.

More precisely, as visible in FIG. 7, a side inlet duct 111, for the intake of supply fluid F′, is situated along the upper holding plate 33, outside it, in the top part of reactor 1. Similarly, a side outlet duct 112, for the extraction of supply fluid F′, is situated along the lower holding plate 33, outside it, in the bottom part of reactor 1.

Thus, the supply fluid F′ is fed and discharged via ducts 111, 112 made outside the tubular holding plates 33, and providing sealed connection with outside of the inlet plenums 41 and outlet plenums 51. In addition, the flow of the supply fluid F′ is directed axially and co-currently to the gas circulation. However, a counter-current concept may also be of interest.

In the exemplary embodiment of FIGS. 7 and 8, the supply chambers 100 are fed via additional tappings in the inlet 42 and outlet 51 plenums.

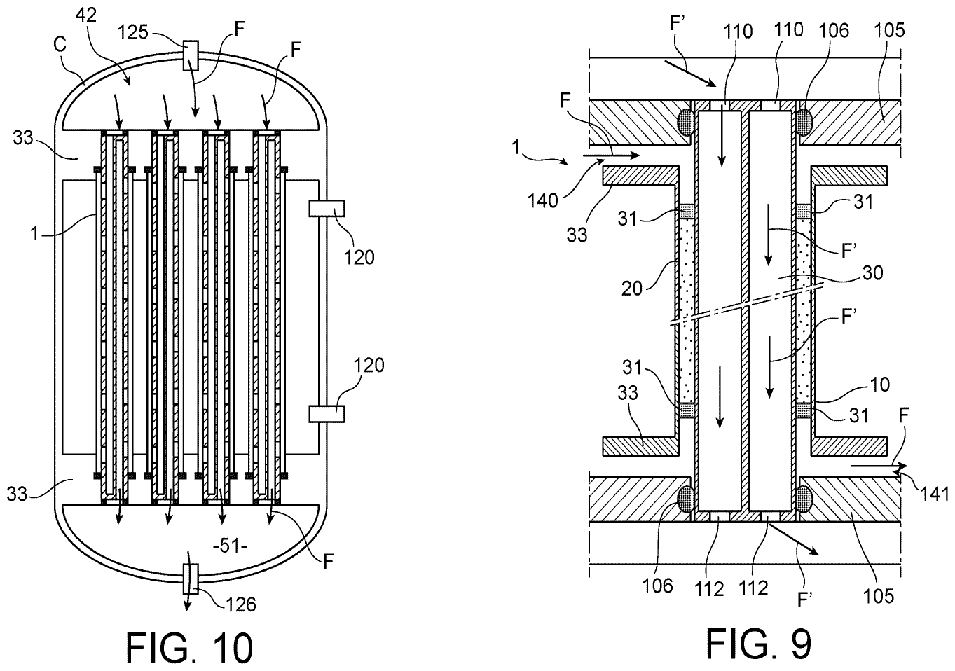

More precisely, as is visible in FIG. 7, supply fluid F′ is fed and extracted via the upper and lower ends of the hollow insert 20. The reactive fluids F are then laterally introduced via side reactive fluid intake 140 and extraction 141 ducts, as is visible in FIG. 9. Sealed separation between the supply fluids F′ and the reactive fluids F is ensured by removable separation plates 105, inserted into the inlet and outlet plenums and each disposed around the hollow insert 20. A seal 106 is then placed between each separation plate 105 and the hollow insert 20.

Advantageously, the one embodiments of FIGS. 5 and 6, on the one hand, and FIGS. 7 and 8, on the other hand, enable a tubular plate/tube assembly close to conventional embodiments.

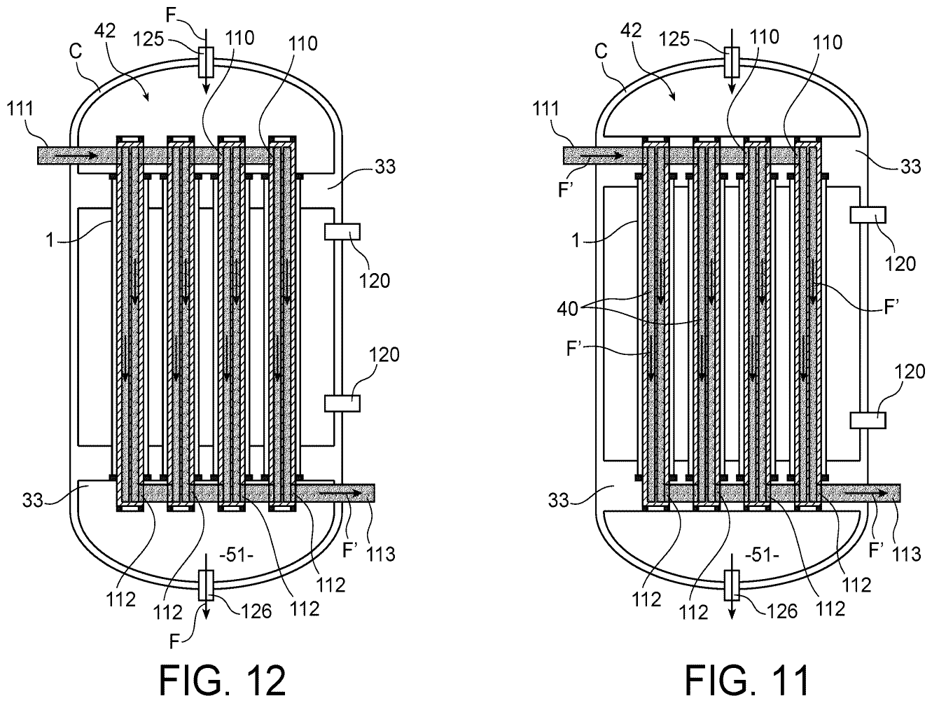

FIGS. 10 and 11 illustrate the implementation of a plurality of tubular reactors 1 in accordance with the invention, especially according to the alternative of FIGS. 1 to 5. Similarly, FIG. 12 illustrates the implementation of a plurality of tubular reactors 1 in accordance with the invention according to the alternative of FIGS. 6 and 7.

This implementation especially comprises four tubes 1 disposed in parallel to one another in a calender C. Tubular holding plates 33 are used to hold the tubes 1 and to provide a circulation space for a heat transfer fluid intended to cool the tubes 1, by means of heat transfer fluid feed and discharge systems 120.

In the example of FIGS. 10 and 11, the distribution chamber or chambers 40 are directly fed from the inlet plenum 42 via reactant feed systems 125. And then, the unconverted products and reactant are discharged into the outlet plenum 51 via extraction systems 126.

Here, the outer tubes are shorter than the inserts 20, and the supply fluid F′ is directly distributed into the inserts 20 via ducts 111, 113 integrated into the tubular holding plates 33.

In the example of FIG. 12, the hollow inserts 20 are longer. The supply fluid F′ is fed and extracted by a dedicated circuit of ducts 111, 113 situated outside the holding plates 33.

The tubular reactor 1 according to the present invention, and especially the implementation of supply chambers 100 for circulating utility or reactive fluids, makes it possible to extend thermalisation capacities.

This configuration moreover favourably responds to the problem of heating the catalyst powder, especially a catalyst in powder form, and thus limits the appearance of hot spots. The result is a more efficient and longer-lasting device. Furthermore, the arrangement of the catalytic powder in the annular space 30 facilitates its cooling.

### Application Example: Application to the Synthesis of Methanol (MeOH)

Two numerical models have been carried out on the principle of the invention using a permselective membrane 160, with the target being the direct synthesis of methanol (MeOH) from carbon dioxide (CO₂).

Only one tube section has been modelled, in a configuration consistent with the alternative of FIG. 2B. The simulation conditions were: a pressure (P) of 50 bar, and a reactant inlet temperature (T) of 200° C., for stoichiometric inlet conditions for this reaction.

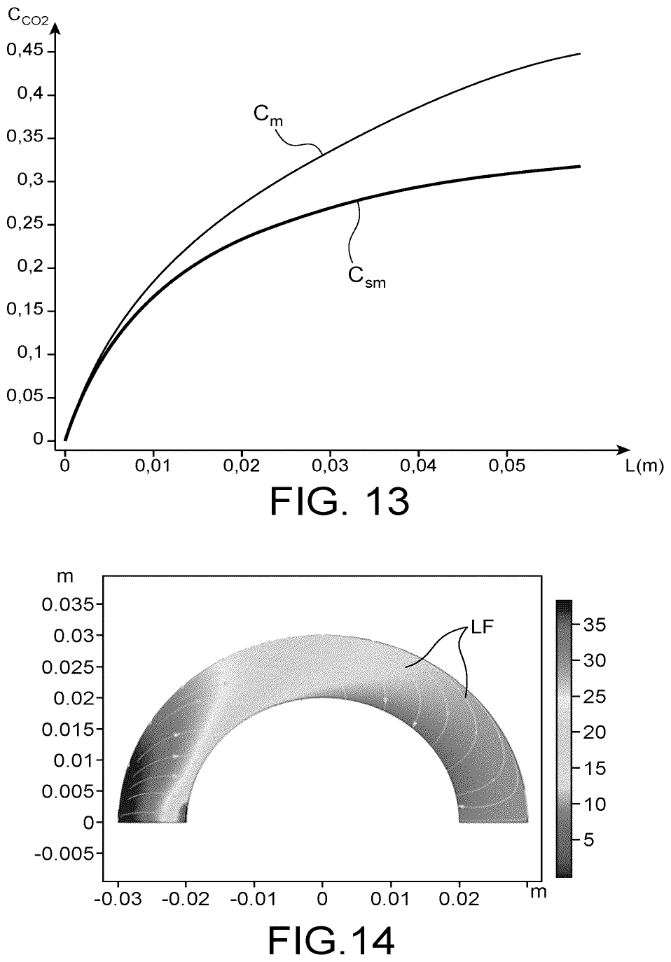

The results obtained are especially visible on the graph in FIG. 13, which represents the course of the conversion of carbon dioxide (C_{CO2}) as a function of the length L, expressed in metres (m), the arc length of the insert 20, in a configuration with the presence of a membrane (Cm) and in a configuration without the presence of a membrane (Cₛₘ), at 200° C. and 50 bar, and on the graph in FIG. 14 which represents a cross-section of the catalyst showing the course of the molar concentrations m, expressed in mol/m³, for the species H₂O and the LF lines of total water flow.

The results show an increased conversion rate as a result of the separating action of the membranes 160, and according to the flow regimes, a depletion in H₂O concentration in the reaction products visible throughout the thickness of the catalyst.

Of course, the invention is not limited to the exemplary embodiments just described. Various modifications may be made by the person skilled in the art.

In particular, the number, and the respective and angular arrangement, of the supply 100, collection 50 and distribution 40 chambers may vary.

The direction of circulation of the supply fluids F′ in the supply chambers 100 can vary.

The choice of location for the feeds and extractions, especially at the end of insert 20 or by side tapping of insert 20, of supply fluids F′ and reactants F respectively may vary according to the method.

Feed to the supply chambers 100 may or may not be associated with the tubular plates 33.

Reactor 1 according to the invention may or may not comprise staggered injection means.

In case the supply chambers 100 are used as cooling chambers, efficiency of the system can be further improved by various manipulations aiming at boosting heat exchanges and known to the person skilled in the art, such as structuring, especially micro-structuring, of surfaces, modifications to the thermal-hydraulic regimes, among other things.

1. A fixed-bed tubular reactor which extends,
along a longitudinal axis, between a first end and a second end, the reactor comprising a catalytic powder bed confined in an annular space situated between an outer wall of a hollow tube and an inner wall of a hollow insert disposed coaxially in the hollow tube,
the hollow insert comprising at least one distribution chamber and at least one collection chamber, separated from each other by at least one first separative wall,
the at least one distribution chamber and the at least one collection chamber comprising a gas intake opening at the first end [(11)] and a gas discharge opening at the second end respectively,
wherein the inner wall of the hollow insert is covered with a separative structure (160, 170) comprising at least one permselective membrane for partially removing at least one reaction product, so that the annular space is delimited by the outer wall and the permselective membrane, and
wherein the reactor further comprises at least one supply chamber separated from the at least one distribution chamber and from the at least one collection chamber by at least one first separative wall, the at least one supply chamber comprising an inlet port for at least one supply fluid, consisting of a flushing fluid for discharging the at least one reaction product, distinct from the gases circulating in the at least one distribution chamber and in the at least one collection chamber, at the first end, and an outlet port for the at least one supply fluid, at the second end.

2. The reactor of claim 1, wherein the permeability of the permselective membrane is selectively exerted with respect to water vapour.

3. The reactor of claim 1, wherein the separative structure additionally includes a porous support covering the inner wall of the insert, the porous support itself being covered with the permselective membrane, and
wherein the hollow insert includes apertures to allow the at least one reaction product to be separated from the gases to pass therethrough.

4. The reactor of claim 1, wherein the hollow insert is made of a porous material, to allow the at least one reaction product to be separated from the gases to pass therethrough, and is covered with the permselective membrane, and
wherein the at least one first separative wall of the hollow insert includes a sealing material for sealing with respect to the reactive gases.

5. The reactor of claim 1, further comprising at the first end and at the second end, respectively, a distributing space and a collecting space between which the hollow insert is disposed.

6. The reactor of claim 1, wherein the tube and the insert are held by at least two tubular holding plates at the first and second ends respectively, at least one of the at least two tubular holding plates including an inlet or outlet duct respectively fluidically connected to an inlet port or outlet port for supply fluid and formed in the at least one of the at least two tubular holding plates, to allow side intake and/or extraction of supply fluid.

7. The reactor of claim 5, wherein the tube and the insert are held by at least two tubular holding plates respectively at the first and second ends, and
wherein at least one inlet or outlet duct respectively fluidically connected to an inlet port or outlet port for supply fluid is situated in the distributing space and in the collecting space respectively, outside the at least two tubular holding plates, to allow side intake and/or extraction of supply fluid.

8. The reactor of claim 5, wherein the tube and the insert are held by at least two tubular holding plates at the first and second ends respectively, and
wherein the inlet port and the outlet port for the at least one supply fluid are respectively formed in the upper and lower ends of the insert for feeding from the distributing space and extracting from the collecting space respectively, the reactor further comprising at least one side gas feed duct and at least one side gas extraction duct, the at least one side feed duct and the feed of fluid supply from the distributing space on the one hand, and the at least one side extraction duct and the extraction of supply fluid from the collecting space on the other hand, being especially separated by a sealed separation plate.

9. The reactor of claim 8, wherein a seal is disposed between the insert and each of the sealed separation plates.

10. The reactor of claim 1, wherein the flow of the at least one supply fluid is made co-currently or counter-currently to the flow of gases in the at least one distribution and collection chamber.

11. The reactor of claim 1, wherein the catalytic powder is retained in the annular space by a seal of fibrous material at each of the ends of the annular space.

12. The reactor of claim 1, wherein the insert is a one-piece part.

13. A method of synthesizing a fuel or a combustible, the method comprising:
implementing an endothermic or exothermic reaction within the fixed-bed tubular reactor of claim 1.
